(19) United States
(12) Patent Application Publication
(10) Pub. No.: US 2015/0012113 A1
(43) Pub. Date: Jan. 8, 2015

(54) APPARATUS FOR COUPLING PROSTHETIC COMPONENTS HAVING ANGULAR AND TRANSLATIONAL OFFSET

(71) Applicant: Medex International, Inc., Bethesda, MD (US)

(72) Inventor: DOGAN CELEBI, Bethesda, MD (US)

(21) Appl. No.: 14/323,679

(22) Filed: Jul. 3, 2014

Related U.S. Application Data
(60) Provisional application No. 61/842,738, filed on Jul. 3, 2013.

(51) Int. Cl. A61F 2/78

(52) U.S. Cl. CPC A61F 2/7812; A61F 2002/7887
USPC 623/38

(57) ABSTRACT

An prosthetic alignment device. In one embodiment, the prosthetic alignment device includes a first component having a first end portion configured to be coupled to one of a prosthesis or a residual limb and a second end portion having a curved channel extending along a curved channel axis; a second component extending along a second central axis comprising a first end portion configured to be coupled to the other one of the prosthesis or the residual limb and a second end portion comprising a curved bar that extends along a curved bar axis, the curved bar positioned within the curved channel so as to be slidable along the curved channel axis in first and second opposite directions so that the first and second components can be selectively adjusted to a plurality of relative positions.

### CROSS-REFERENCE TO RELATED APPLICATIONS

This application claims priority to U.S. Provisional Patent Application Ser. No. 61/842,738, filed on Jul. 3, 2013, the entirety of which is incorporated herein by reference.

### FIELD OF THE INVENTION

This invention relates generally to an apparatus for connecting prosthetic components, and specifically to an apparatus for coupling prosthetic components that allows for both angular and translational offset therebetween.

### BACKGROUND OF THE INVENTION

Various types of foot and leg prosthetic devices are well known in the art. Such devices frequently include some form of attachment for coupling the device to the distal end of the limb of the amputee and for extending to the ground to provide body support. For reasons understood in the art, it is often desirable for a user to be able to adjust the position and/or angle of the artificial limb to the residual limb (or stump). This is particularly desirable in the case of a prosthetic leg and foot combination wherein it is desirable to adjust the position of the leg and foot combination relative to the residual limb to achieve optimum comfort in different use situations.

Prosthetic coupling devices that allow for offset alignment are known in the art. An example of an existing offset alignment device is disclosed in U.S. Pat. No. 7,090,700, issued Aug. 15, 2006 to Curtis, the teachings of which are incorporated herein by reference. The Curtis offset alignment device has a longitudinal axis that can offset two prosthetic components a selected distance along an alignment axis that is selectably oriented in a plane that is generally perpendicular to the longitudinal axis. As such, the Curtis offset alignment device can also adjust the distance between the two prosthetic components in a direction generally parallel to the longitudinal axis. The Curtis offset alignment device comprises a first member having a channel and a second member having a bar for being adjustably connected to the channel along the alignment axis. A set screw can be inserted through a hole in the wall of the channel in a direction generally perpendicular to the alignment axis to contact the bar and hold it stationary within the channel. One or both of the first and second members can be rotatably connected to one or both of the first and second prosthetic components, respectively.

While the Curtis offset alignment device is suitable in certain regards, it is limited by the size of the first and second members in the amount of offset that can be achieved, thereby limiting the available offset of the prosthetic components. Stated simply, if the first member is translated relative to the second member too far, the bar will come out of the channel, thereby causing the first and second members to come apart. Moreover, because the Curtis offset alignment device only allows the first and second members to be offset relative to one another in a plane that is generally perpendicular to the longitudinal axis of the Curtis offset alignment device, the prosthetic component can only be offset a distance equal to the transverse width of the first or second members at most.

Consequently, there exists a need for a new and improved apparatus for coupling prosthetic components.

### BRIEF SUMMARY OF THE INVENTION

The present invention is directed to a prosthetic alignment device including a first component and a second component that are movably coupled together. The first component has a first end portion for coupling to a prosthesis or a residual limb and a second end portion including a curved channel. The second component has a first end portion for coupling to the other one of the prosthesis or the residual limb and a second end portion having a curved bar. The first and second components are coupled together so that the curved bar is positioned within the curved channel. The curved bar is slidable within the curved channel in first and second opposite directions so that the first and second components can be selectively adjusted to different relative positions.

In one aspect, the invention can be a prosthetic alignment device comprising: a first component extending along a first central axis comprising: a first end portion configured to be coupled to one of a prosthesis or a residual limb; and a second end portion comprising a curved channel extending along a curved channel axis between opposite lateral side surfaces of the first component; a second component extending along a second central axis comprising: a first end portion configured to be coupled to the other one of the prosthesis or the residual limb; and a second end portion comprising a curved bar that extends along a curved bar axis; the curved bar slidably disposed within the curved channel to couple the first and second components together, the curved bar slidable within the curved channel so as to be slid along the curved channel axis in first and second opposite directions so that the first and second components can be selectively adjusted to a plurality of relative positions, the plurality of relative positions comprising: (1) a first relative position in which the first and second central axes are parallel to one another; and (2) a second relative position in which the first and second central axes are angularly offset from one another.

In another aspect, the invention can be a prosthetic alignment device comprising: a first component comprising: a first end portion configured to be coupled to one of a prosthesis or a residual limb; and a second end portion comprising a curved channel extending along a curved channel axis; a second component extending along a second central axis comprising: a first end portion configured to be coupled to the other one of the prosthesis or the residual limb; and a second end portion comprising a curved bar that extends along a curved bar axis; the curved bar positioned within the curved channel so as to be slidable along the curved channel axis in first and second opposite directions so that the first and second components can be selectively adjusted to a plurality of relative positions.

In yet another aspect, the invention can be a prosthetic alignment device comprising: a first monolithic component extending along a first central axis, the first component consisting of: a first end portion configured to be coupled to one of a prosthesis or a residual limb; and a second end portion comprising a curved channel extending along a curved channel axis between opposite lateral side surfaces of the first component; a second monolithic component extending along a second central axis consisting: a first end portion configured to be coupled to the other one of the prosthesis or the residual limb; and a second end portion comprising a curved bar that extends along a curved bar axis; the curved bar slidably disposed within the curved channel to couple the first and second components together, the curved bar slidable within the curved channel so as to be slid along the curved channel axis in first and second opposite directions so that the first and second components can be selectively adjusted to a plurality of relative positions, the plurality of relative positions comprising: (1) a first relative position in which the first and second central axes are parallel to one another; and (2) a second relative position in which the first and second central axes are angularly offset from one another wherein each of the first and second components is a monolithic integrally formed singular component; wherein each of the channel and bar axes have a substantially constant radius of curvature along their entire length; the curved channel axis intersecting the first central axis at a first intersection point, wherein a line tangent to the curved channel axis at the first intersection point is substantially perpendicular to the first central axis; the curved bar axis intersecting the second central axis at a second intersection point, wherein a line tangent to the curved bar axis at the second intersection point is substantially perpendicular to the second central axis; the curved bar has a curved terminal surface and the curved channel has a curved floor surface, wherein the curved terminal surface, the curved floor surface, the curved bar axis, and the curved channel axis have substantially equal radii of curvature; and a locking element operably coupled to the first component, the locking element alterable between: (1) a first state in which the locking element does not prohibit the curved bar from sliding within the curved channel; and (2) a second state in which the locking element locks the first and second components in a selected one of the plurality of relative positions.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

FIG. 1 is a perspective view of an apparatus for coupling prosthetic components comprising a first component and a second component according to one embodiment of the present invention, wherein the second component comprises a pyramid block portion;

FIG. 2 is an exploded view of the apparatus of FIG. 1;

FIG. 3 is a bottom perspective view of the first component of the apparatus of FIG. 1;

FIG. 4A is cross-sectional view of the first component taken along line 4A-4A of FIG. 3;

FIG. 4B is cross-sectional view of the first component taken along line 4B-4B of FIG. 3;

FIG. 5 is a top perspective view of the second component of the apparatus of FIG. 1;

FIG. 6A is cross-sectional view of the first component taken along line 6A-6A of FIG. 5;

FIG. 6B is cross-sectional view of the first component taken along line 6B-6B of FIG. 5;

FIG. 7A is cross-sectional view of the apparatus of FIG. 1 taken along line 7A-7A of FIG. 1;

FIG. 7B is cross-sectional view of the apparatus of FIG. 1 taken along line 7B-7B of FIG. 1;

FIG. 8A is a schematic of the apparatus of FIG. 1 coupling prosthetic components together wherein the second component is in an aligned position with respect to the first component so that the longitudinal axes of the first and second components are substantially coaxial with one another;

FIG. 8B is a schematic of the apparatus of FIG. 1 coupling prosthetic components together wherein the second component is in a first offset position with respect to the first component so that the longitudinal axes of the first and second components are angularly offset with one another;

FIG. 8C is a schematic of the apparatus of FIG. 1 coupling prosthetic components together wherein the second component is in a second offset position with respect to the first component so that the longitudinal axes of the first and second components are angularly offset with one another;

FIG. 9 is a schematic wherein the views of FIGS. 8A-C have been superimposed;

FIG. 10 is a perspective view of an apparatus for coupling prosthetic components comprising a first component and a second component according to a second embodiment of the present invention, wherein the second component comprises a pyramid block receiving portion;

FIG. 11 is an exploded view of the apparatus of FIG. 10;

FIG. 12A is cross-sectional view of the apparatus of FIG. 10 taken along view 12A-12A of FIG. 1;

FIG. 12B is cross-sectional view of the apparatus of FIG. 10 taken along view 12B-12B of FIG. 1; and

FIG. 13 is a schematic cross-sectional view of an apparatus for coupling prosthetic components comprising a first component and a second component according to a third embodiment of the present invention, wherein each of the first and second components comprises a pyramid block receiving portion.

### DETAILED DESCRIPTION OF THE INVENTION

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

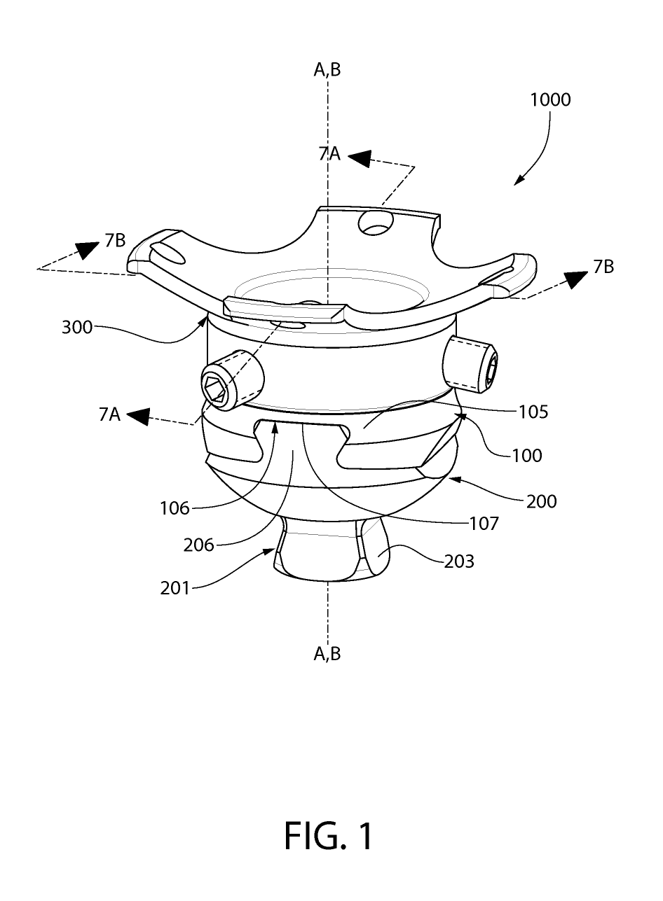

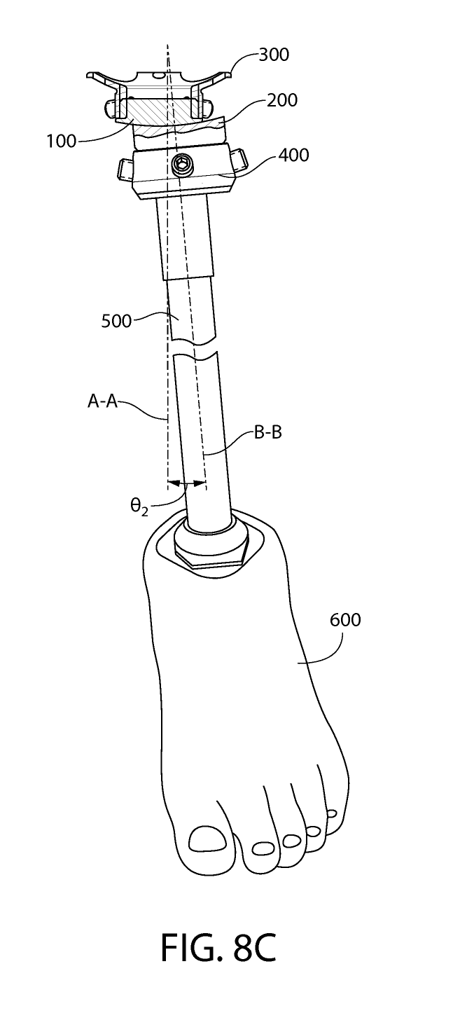

Referring now to FIGS. 1, 2, and 8A concurrently, a prosthetic coupling apparatus 1000 according to one embodiment of the present invention is illustrated. The prosthetic coupling apparatus 1000 is generally used to couple two different prosthetic components together while enabling the two different prosthetic components to be angularly and translationally adjusted relative to one another. Specifically, as best seen in FIG. 8A, the prosthetic coupling apparatus 1000 is used to couple a first prosthetic component (i.e., a cup member 300) to a second prosthetic component (i.e., a pyramid receiver 400 of a pylon 500). Although the cup member 300 and pylon 500 are illustrated herein, the invention is not to be so limited and the prosthetic coupling apparatus 1000 may be used to couple any other type of prosthetic components together as would be understood by persons of ordinary skill in the art. In the exemplified embodiment, the cup member 300 may be coupled to a wearer's residual limb, and the pylon 500, which may form a prosthetic leg, may be coupled to or be formed integrally with a prosthetic foot. U.S. Patent Application Publication No. 20110015761, published Jan. 20, 2011, by the inventor of the present application, provides background for the coupling of a prosthetic foot to a residual limb, and is incorporated herein by reference in its entirety for background in that regard.

The prosthetic coupling apparatus 1000 enables two different prosthetic components to be adjusted relative to one another so as to be angularly and translationally offset to a desired degree. Specifically, the positioning of the two different prosthetic components (i.e., the cup member 300 and the pylon 500) relative to one another may affect the wearer's gait and whether or not the wearer's weight is centered over the lowermost of the prosthetic components, which can negatively affect the wearer's comfort and may compromise or cause failure to the prosthetic limb. Using the prosthetic coupling apparatus 1000, a wearer can adjust the relative positioning of two different prosthetic components to ensure comfort and to enhance the usable lifetime of the prosthetic components.

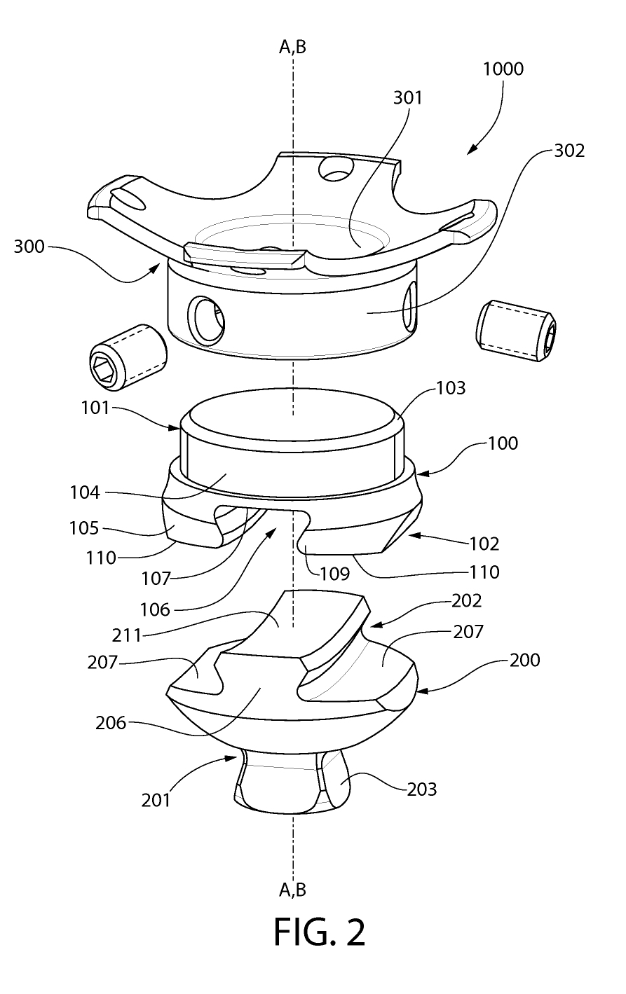

Referring now to FIGS. 1 and 2 concurrently, the prosthetic coupling apparatus 1000 will be described in detail. The prosthetic coupling apparatus 1000 generally comprises a first component 100 and a second component 200 that are detachably coupled together to form the prosthetic coupling apparatus 1000. In FIGS. 1 and 2, the cup member 300 is shown coupled to the first component 100 and the pyramid receiver 400 of the pylon 500 is shown coupled to the second component 200. However, in other embodiments the cup member 300 may be coupled to the second component 200 and the pyramid receiver 400 of the pylon 500 may be coupled to the first component 100. In certain embodiments, one of the first and second components 100, 200 is coupled to one of a prosthesis (i.e., the pyramid receiver 400 and pylon 500) or a residual limb (i.e., the cup member 300 which is coupled to the residual limb) and the other of the first and second components is coupled to the other one of the prosthesis or the residual limb. Thus, the invention is not to be limited by the specific prosthetic components to which each of the first and second components 100, 200 is attached, and various modifications in this regard are within the scope of the present invention.

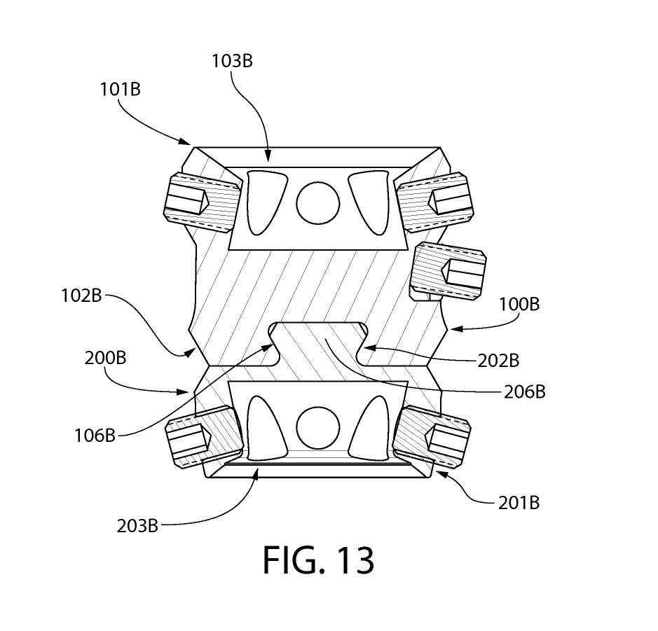

Furthermore, although depicted in the exemplary embodiments, it should be appreciated that the cup member 300 does not form part of the prosthetic coupling apparatus 1000 in all embodiments and may be omitted and/or replaced with other prosthetic components as appropriate and depending on the use of the prosthetic coupling apparatus 1000. One particular embodiment in which the cup member 300 is omitted will be described with reference to FIG. 13, in which a body having a pyramid receiving cavity is used instead of the cup member 300. As described in greater detail below, the prosthetic coupling apparatus 1000 is used to couple prosthetic components together (exemplified as the cup member 300 and the pyramid receiver 400 of the pylon 500 in FIGS. 8A-8C, although the cup member 300 and the pyramid receiver 400/pylon 500 can be exchanged for other prosthetic components as desired).

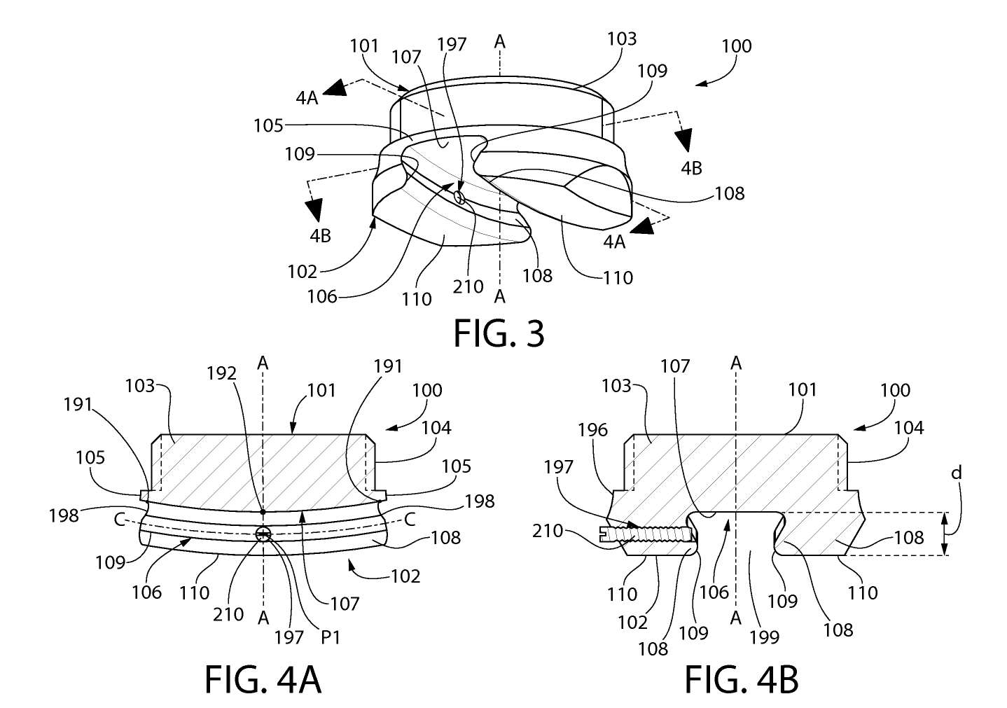

Referring now to FIGS. 1-4B concurrently, the first component 100 of the prosthetic coupling apparatus 1000 will be further described. The first component 100 extends along a first central axis A-A from a first end portion 101 to a second end portion 102. Furthermore, the first component 100 has opposing sidewalls 105 and opposing sidewalls 196. In the exemplified embodiment, the opposing sidewalls 105, 196 form a single outer surface of the first component 100, particularly in the exemplified embodiment in which the first component 100 has a circular or rounded transverse cross-sectional shape. Thus the opposing sidewalls 105, 196 form portions of and collectively define the outer surface of the first component 100.

In the exemplified embodiment, the first component 100 is a unitary, monolithic structure that extends from the first end portion 101 to the second end portion 102. Thus, the first component 100 is a monolithic integrally formed singular component. In the exemplified embodiment, the first end portion 101 of the first component 100 is configured to be coupled to a prosthetic component (such as the cup member 300 depicted in FIGS. 1 and 2). Furthermore, the first end portion 101 of the first component 100 comprises a hub 103 having an outer surface 104. In certain embodiments, the outer surface 104 of the hub 103 may be threaded so that the outer surface 104 of the hub 103 can threadily engage a threaded inner surface 301 of a collar 302 of the cup member 300. In other embodiments, the hub 103 may have a smooth outer surface and/or may be an annular structure having a threaded inner surface.

In still other embodiments, the first end portion 101 of the first component 100 may take on other structural configurations that are designed to couple with a different prosthetic component. In such an embodiment, the hub 103 may be supplemented and/or supplanted by other structures. For example, in some embodiments the first end portion 101 of the first component 100 may comprises a pyramid block, a pyramid receiving cavity, an annular collar, a cup, or the like (one example of which is described in more detail below with reference to FIG. 13 in which the hub has been replaced by a pyramid receiving cavity) to enable the first component 100 to be coupled to different types of prosthetic components.

The second end portion 102 of the first component 100 comprises a curved channel 106 that extends through the first component 100 from the opposite lateral sides 105 of the first component 100 along a curved channel axis C-C. Thus, the curved channel 106 terminates at an opening 198 on both of the opposite lateral sides 105 of the first component 100. Furthermore, the curved channel 106 extends a depth d into the first component 100 from a terminal end of the second end portion 102 of the first component 100 in a direction of the first end portion 101 of the first component 100. Thus, an elongated opening 199 is formed at the second end portion 102 of the first component 100 that forms a passageway into the curved channel 106, the elongated opening 199 extending between the opposite lateral sides 105 of the first component 100.

The curved channel 106 is defined by opposing curved walls 108 and a curved floor surface 107. In the exemplified embodiment, the curved floor surface 107 is curved or arcuate so as to define a convex surface. Specifically, the curved floor surface 107 has low points 191 located at the opposing lateral sides 105 and a high point 192 located centrally between the opposing lateral sides 105. Similarly, the curved walls 108 have terminal surfaces 110 that are curved or arcuate so that the second end portion 102 of the first component 100 defines or forms a convex surface. The curved walls 108 have high and low points that are transversely aligned (relative to the curved channel axis C-C) with the low and high points 191, 192 of the curved floor surface 107. The invention is not to be limited to the specific structural arrangement depicted in the figures in all embodiments, and the convex surfaces can be concave surfaces in some other embodiments. Specifically, as discussed in more detail below, in the exemplified embodiment the first component 100 has convex surfaces and the second component 200 has concave surfaces that mate with the convex surfaces of the first component 100. However, the invention is not to be so limited and the first component 100 may have concave surfaces that mate with convex surfaces of the second component 200 in other embodiments.

Thus, in the exemplified embodiment the curved channel axis C-C is convex when viewed from the second end portion 102 of the first component 100 and concave when viewed from the first end portion 101 of the first component 100. Furthermore, the cross-sectional area of the curved channel 106 is substantially constant along the length of the curved channel 106 from the opposing lateral ends 105 of the first component 100. Specifically, the radii of curvature of the terminal surfaces 110 of the curved walls 108 and the curved floor 107 are substantially the same so that the cross-sectional area of the curved channel 106 remains substantially constant along its length.

In the exemplified embodiment the curved channel axis C-C has a substantially constant radius of curvature along its entire length. Similarly, in the exemplified embodiment the curved floor surface 107 also has a substantially constant radius of curvature that is substantially equal to that of the curved channel axis C-C. Further still, the terminal surfaces 110 of the curved walls 108 also have a substantially constant radius of curvature that is substantially equal to that of the curved floor 107 and of the curved channel axis C-C. However, the invention is not to be so limited in all embodiments and the radius of curvatures of the curved channel axis C-C, the curved floor surface 107, and the terminal surfaces 110 of the curved walls 108 may be non-constant in other embodiments. Furthermore, in some embodiments, regardless of whether the radii of curvature of the curved channel axis C-C, the curved floor surface 107, and the terminal surfaces 110 of the curved walls 108 are constant, they may be different from one another in some embodiments.

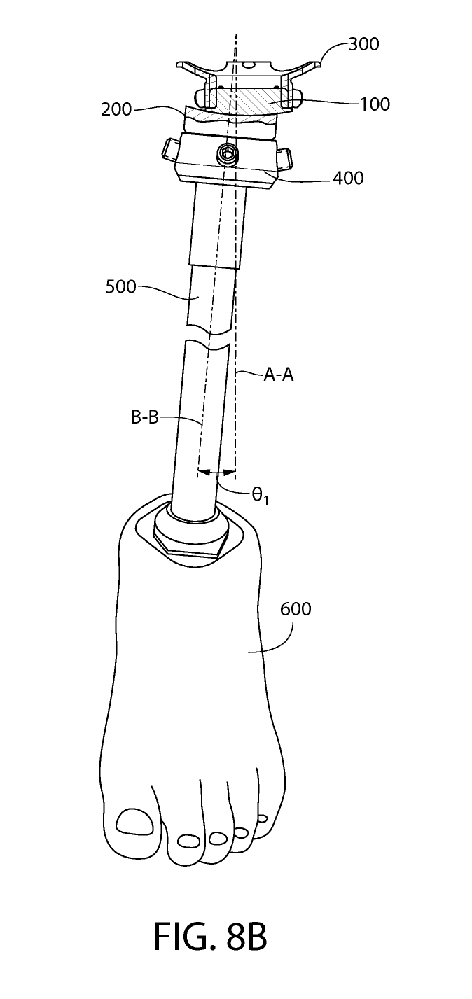

Each of the curved walls 108 that assists in defining the curved channel 106 comprise a flange portion 109 that protrudes into the curved channel 106. Thus, the curved channel 106, in the exemplified embodiment, has a dovetail cross-sectional profile (taken transverse to the curved channel axis C-C). Furthermore, as discussed in more detail below with reference to FIGS. 7A-8C, the curved channel 106 of the first component 100 slidably receives a curved bar 206 of the second component 200 to detachably couple the first and second members 100, 200 of the prosthetic coupling apparatus 1000 together.

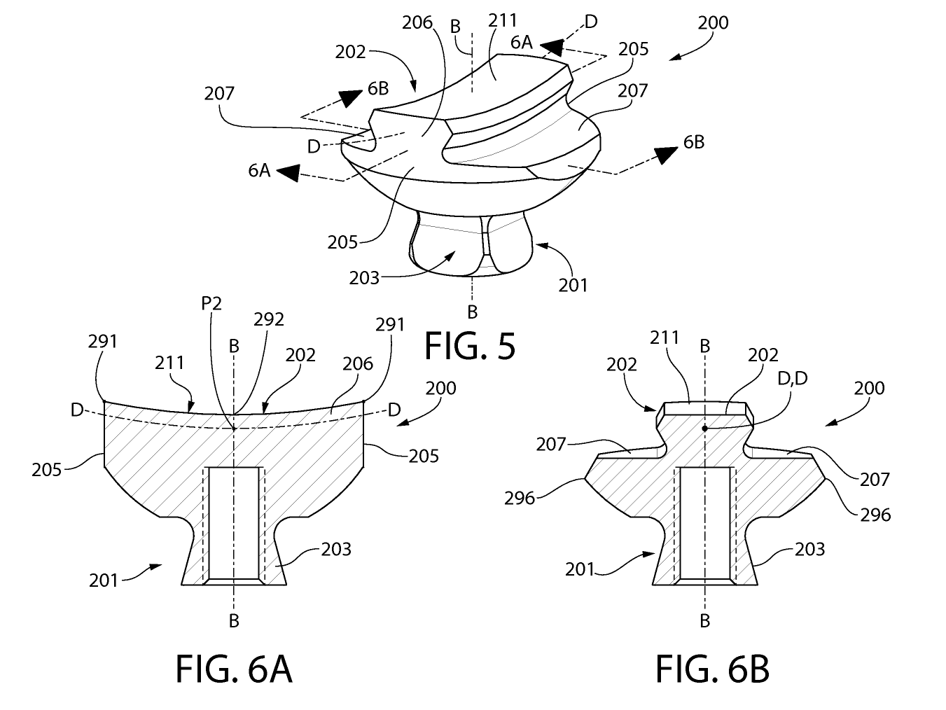

As can be seen in FIG. 4A, the curved channel axis C-C intersects the first central axis A-A at a first intersection point P1. In the exemplified embodiment, a line tangent to the curved channel axis C-C at the first intersection point P1 is substantially perpendicular to the first central axis A-A.

In the exemplified embodiment, the first member 107 has a through hole 197 extending through at least one of the opposing sidewalls 196. Thus, the curved channel 106 extends between the opposing sidewalls 105 and the through hole 197 is formed into at least one of the other opposing sidewalls 196. In the exemplified embodiment, a single through hole 197 is illustrated being formed into only one of the opposing sidewalls 196. However, in other embodiments multiple through holes 197 (such as two, three, four, or more through holes) can be formed into one of the opposing sidewalls 196, one through hole 197 can be formed into each of the opposing sidewalls 196, or multiple through holes 197 can be formed into each of the opposing sidewalls 196 as desired. Each of the through holes 197 extends through the first component 100 from the sidewall 196 to the curved channel 106 thereby forming a passageway from the exterior environment to the curved channel 106 through the first component 100. In the exemplified embodiment, a single through hole 197 is illustrated being centered along the curved channel 206; however, when multiple through holes 197 are used, they can be positioned as desired along the curved channel 206 in any manner that is deemed sufficient to secure the second component 200 to the first component 100 with set screws 210 positioned within the through holes 197.

In the exemplified embodiment, a set screw 210 is positioned within the through hole 197. Although illustrated herein as being a set screw 210, in certain embodiments the set screw 210 may be any type of locking element. Thus, although set screws are described herein, the locking element may be a set screw, a fastener, a clamp, a mechanical slideswitch, a depressible locking button, or the like. In embodiments that include more than one of the through holes 197, one set screw is positioned within each of the through holes 197. The set screw 210 has a sufficient length so that the set screw 210 can be positioned within the through hole 197 and so that a terminal end of the set screw 210 penetrates into the curved channel 106 when the set screw 210 is sufficiently screwed into the through hole 197. As a result, the set screw 210 can assist in detachably securing the second component 200 to the first component 100 as will be described in more detail below. Specifically, when the first and second members 100, 200 are coupled together, a curved bar 206 of the second component 200 is positioned within the curved channel 106, and the set screw(s) 210/locking element secure the curved bar 206 within the curved channel 106. This coupling of the first and second members 100, 200 will be discussed in more detail below with reference to FIGS. 7A-8C.

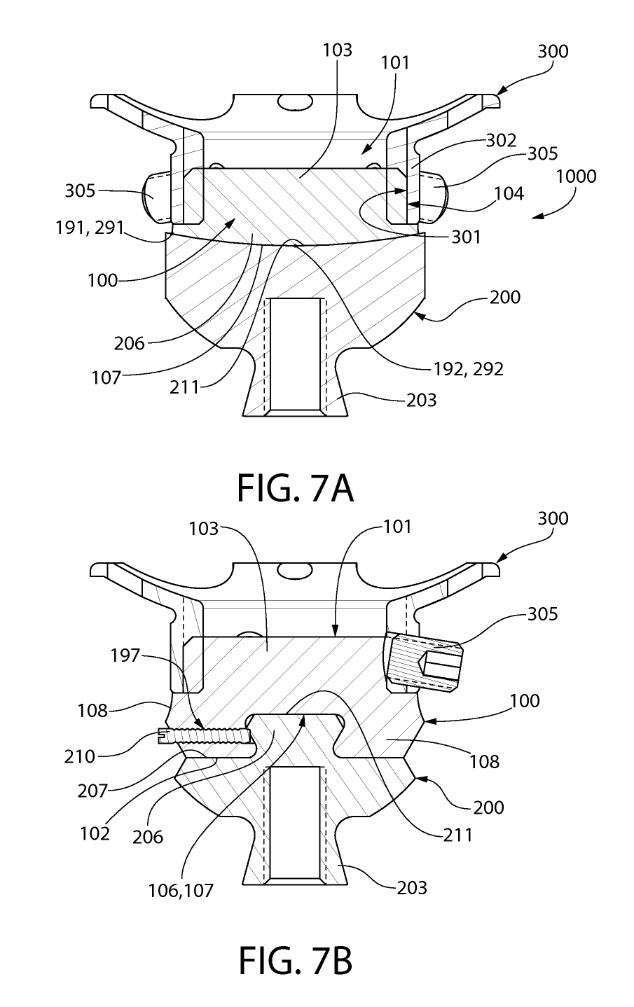

Referring now to FIGS. 1, 2 and 5-6B concurrently, the second component 200 of the prosthetic coupling apparatus 1000 will be described. The second component 200 of the prosthetic coupling apparatus 1000 extends along a second central axis B-B from a first end portion 201 to a second end portion 202. Furthermore, the second component 200 has opposing sidewalls 205 and opposing sidewalls 296. The opposing sidewalls 205, 296 may collectively form a single outer surface of the second component 200, particularly in embodiments as exemplified in which the second component 200 has a circular or rounded transverse cross-sectional shape. In the exemplified embodiment, the second component 200 is a unitary, monolithic structure that extends from the first end portion 201 to the second end portion 202. Thus, the second component 200 is a monolithic integrally formed singular component.

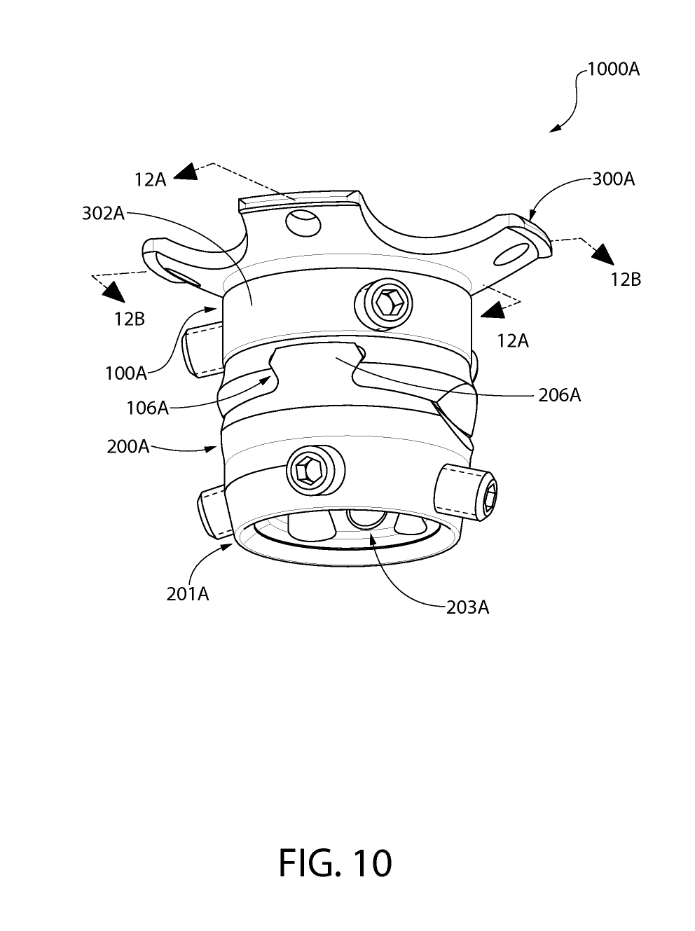

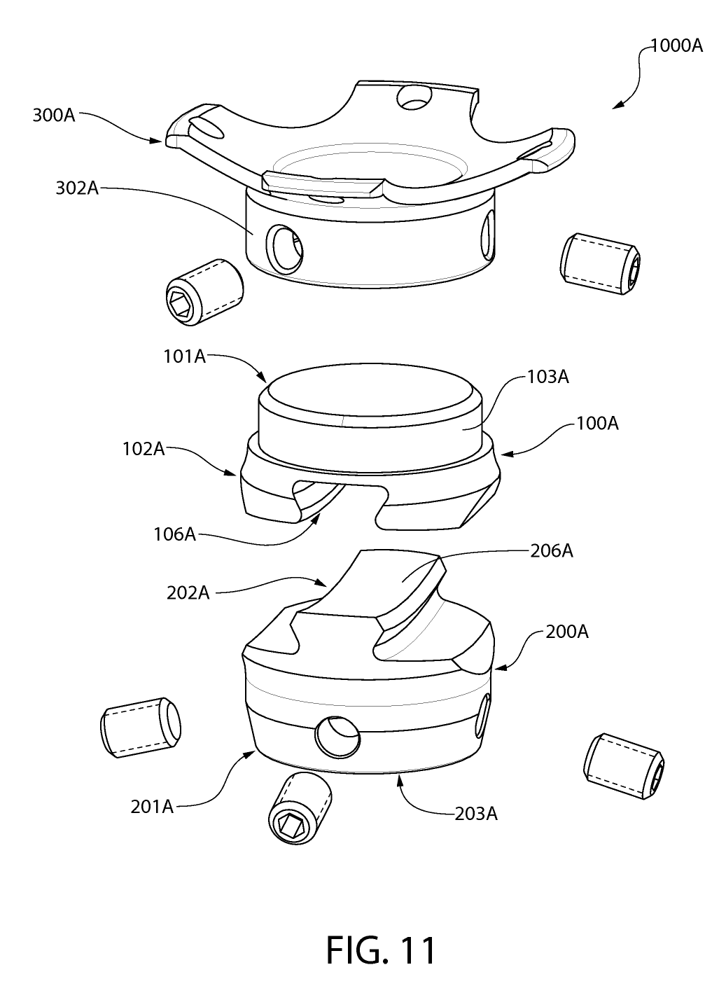

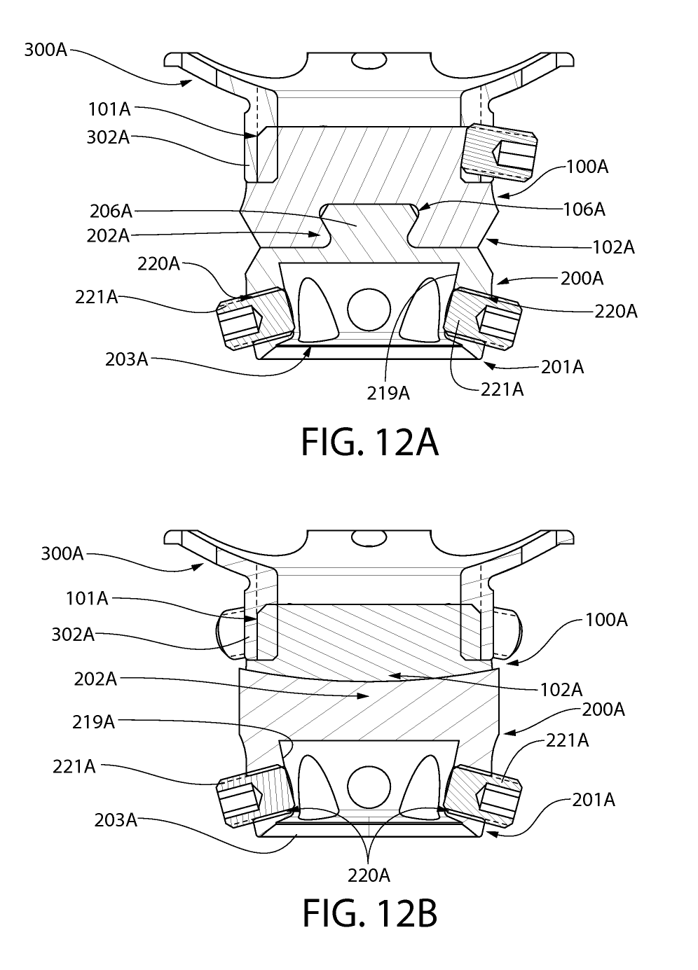

The first end portion 201 of the second component 200 is configured to be coupled to a prosthetic component (such as the pyramid receiver 400 of FIGS. 8A-C, although other prosthetic components may be coupled to the first end portion 201 as would be appreciated by persons of ordinary skill in the art). In the exemplified embodiment, the first end portion 201 of the second component 200 comprises a pyramid block 203. In still other embodiments, the first end portion 201 may take on other structural configurations that are designed to couple with a different prosthetic component. In such an embodiment, the pyramid block 203 may be supplemented and/or supplanted by other structures. For example, the first end portion 201 may comprise a pyramid receiver 203A (see FIGS. 10-12B, described in more detail below). In still other embodiments, the first end portion 201 may comprise a threaded hub, an annular collar, a cup, or the like.

The second component 200 has a curved end surface 207 located between the first and second end portions 201, 202 of the second component 200 in the direction of the second central axis B-B. In the exemplified embodiment, the curved end surface 207 is concave such that the curved end surface 207 has high points at the opposing lateral sides 205 and a low point in between the opposing lateral sides 205. Furthermore, as discussed above the second end 102 of the first component 100 (and the terminal end 110 of the curved wall 108) is convex. In some embodiments the curved end surface 207 has substantially the same radius of curvature as the second end 102 of the first component 100 (and of the terminal end 110 of the curved wall 108). This is desirable because the curved end surface 207 of the second component 200 is in surface contact or at least facing relation with the second end 102 of the first component 100 when the first and second members 100, 200 are coupled together (see FIGS. 7A and 7B, discussed in more detail below). Thus, forming the curved end surface 207 of the second component 200 and the second end 102 of the first component 100 with the same radius of curvature ensures conformal surface contact between those two surfaces when the first and second members 100, 200 are coupled together.

The second end portion 202 of the second component 200 comprises a curved bar 206 that protrudes from the curved end surface 207 of the second component 200. The curved bar 206 extends along the second component 200 between the opposite lateral sides 205 of the second component 200 along a curved bar axis D-D. The curved bar 206 has a width that is less than a width of the second component 200 between the opposing lateral sides 296 of the second component 200. Thus, the curved bar 206 extends the entire length between the opposing lateral sides 205 of the second component 200, but not the entire width between the opposing lateral sides 296 of the second component 200.

The curved bar axis D-D, in one embodiment, has a substantially constant radius of curvature. Furthermore, in certain embodiments the curved bar axis D-D and the curved channel axis C-C have the same radius of curvature to effectuate slidable mating between the curved bar 206 and the curved channel 106 as described in greater detail below. The curved bar 206, in the exemplified embodiment, has a cross-sectional profile (taken transverse to the curved bar axis D-D) that is substantially constant and substantially the same as that of the cross-sectional profile of the curved channel 106. Thus, in the illustrated embodiment the curved bar 206 has a dovetail transverse cross-sectional profile.

The curved bar 206 terminates in a terminal surface 211 that forms an end surface of the second end portion 202 of the second component 200. In the exemplified embodiment, the terminal surface 211 of the curved bar 206 is also curved and, in one embodiment, has a radius of curvature that is substantially the same as that of the curved bar axis D-D. In one embodiment, the curved end surface 207 also has a substantially constant radius of curvature that is substantially equal to that of the curved bar axis D-D. Furthermore, in certain embodiments the terminal surfaces 110 of the curved walls 108 are curved so as to have a substantially constant radius of curvature that is equal to the radius of curvature of the curved end surface 207. In one embodiment, the terminal surface 211 of the curved bar 206, the floor surface 107 of the curved channel 106, the curved bar axis D-D, and the curved channel axis C-C have substantially equal radii of curvature. Furthermore, in some embodiments the terminal surfaces 110 of the curved walls 108 and the curved end surface 207 of the second end portion 202 of the second component 200 have substantially equal radii of curvature.

In the exemplified embodiment, the terminal end 211 of the curved bar 206 forms a concave surface such that the curved bar 206 has high points 291 located at the opposing lateral sides 205 of the second component 200 and a low point 292 located centrally between the opposing lateral sides 205 of the second component 200. Furthermore, as discussed in detail above, the floor surface 107 of the curved channel 106 forms a convex surface and has low points 191 located at the opposing lateral sides 105 of the first component 100 and a high point 192 located centrally between the opposing lateral sides 105 of the first component 100. Furthermore, the radius of curvature of the terminal end 211 of the curved bar 206 is substantially the same as the radius of curvature of the floor surface 107 of the curved channel 106 of the first component 100 so that when the first and second members 100, 200 are coupled together, the terminal end 211 of the curved bar 206 is in substantial conformal surface contact with the floor surface 107 of the curved channel 106.

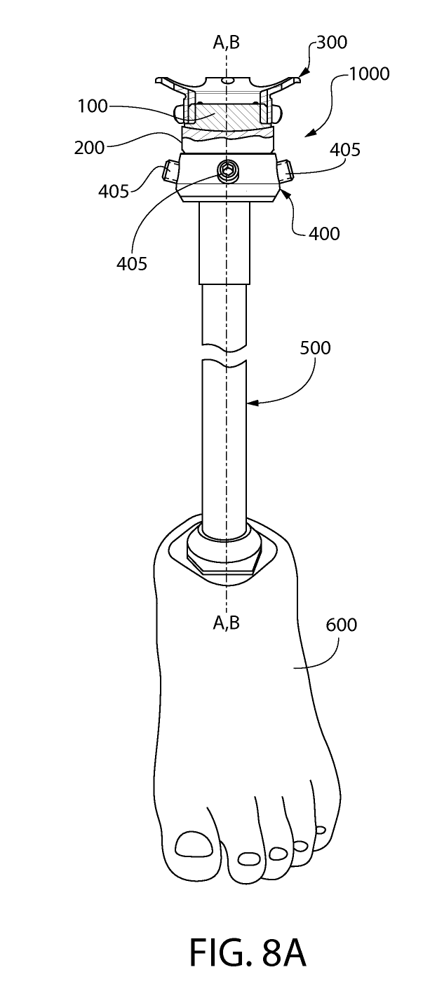

As can be seen in FIG. 6A, the curved bar axis D-D intersects the second central axis B-B at a second intersection point P2. In the exemplified embodiment, a line tangent to the curved bar axis D-D at the second intersection point P2 is substantially perpendicular to the second central axis B-B.

Referring now to FIGS. 7A-9 concurrently, the assemblage and operation of the prosthetic coupling apparatus 1000 to couple prosthetic components will be described. In the exemplified embodiment, the first end portion 101 of the first component 100 is coupled to the first prosthetic component 300 (which is the cup member). Specifically, in the exemplified embodiment, the hub 103 is inserted into the annular collar 302 of the first prosthetic component 300 and coupled thereto via threaded engagement between the outer surface 104 of the hub 103 and the inner surface 301 of the cup member 300. In other embodiments, the threads may be omitted and the hub 103 will simply be slid into the collar 302. Once the hub 103 is in the collar 302, set screws 305 are inserted into through holes that are formed into the cup member 300, and the set screws 305 are tightened, thereby preventing relative rotation between the first component 100 and the first prosthetic component 300. Of course, any type of coupling structure can be utilized and will be dictated on specific needs/uses. Thus, the cup member 300 is simply one example of a first prosthetic component that may be coupled to the first component 100, but other first prosthetic components can be used depending on specific needs and uses.

Turning to the second component 200, the first end portion 201 of the second component 200 is coupled to the second prosthetic component 400 (which in the exemplified embodiment is the pyramid receiver 400 of the pylon 500). Although exemplified herein as being the pyramid receiver 400 of the pylon 500, the second prosthetic component 400 can also be any other type of prosthetic component depending on specific needs and uses. As described in more detail below, in one embodiment the first end portion 201 of the second component 200 may form a pyramid receiver that is coupled to a pyramid of a second prosthetic component. In the exemplified embodiment, coupling of the second component 200 to the pyramid receiver 400 of the pylon 500 is accomplished by inserting the pyramid block 203 of the first member into a pyramid block receiving cavity of the pyramid receiver 400. Next, set screws 405 are inserted into through holes in the pyramid receiver 400 and tightened to prohibit relative movement between the second component 200 and the pyramid receiver 400. Of course, any type of coupling structure can be utilized and will be dictated on specific needs/uses. Thus, the pyramid receiver 400/pylon 500 is simply one example of a second prosthetic component that may be coupled to the second component 200, but other second prosthetic components can be used depending in specific needs and uses.

The first and second members 100, 200 are then coupled together by aligning the curved bar 206 of the second component 200 with the curved channel 106 of the first component 100 and sliding the curved bar 206 into the curved channel 106. Of course, in other embodiments the first and second members 100, 200 can first be coupled together, and then coupled to the other prosthetic components in the manner described herein above. Coupling of the first and second members 100, 200 together requires aligning the curved bar 206 of the second component 200 with one of the openings 198 in one of the opposing lateral sides 105 of the first component 100, and then translating the first and second members 100, 200 relative to one another (in the direction of the curved channel axis C-C) so that the curved bar 206 of the second component 200 is slidably inserted into the curved groove 106 of the first component 100. As a result of this slidable insertion, the curved bar 206 becomes located within and nests within the curved channel 106, thereby movably coupling the first component 100 to the second component 200.

The curved bar 206 has a transverse cross-sectional profile that mates with the transverse-cross sectional profile of the curved channel 106 so that the first and second components 100, 200 cannot be separated from one another without sliding the curved bar 206 along the curved channel axis C-C until the curved bar 206 is located entirely outside of the curved channel 106. Specifically, due to the dove-tail shapes of the curved bar 206 and the curved channel 106, the curved bar 206 cannot be separated from the curved channel 106 in a direction perpendicular to the curved channel axis C-C when the curved bar 206 is located within the curved channel 106. Once the curved bar 206 is located within the curved channel 106, the first and second components 100, 200 can only be separated from one another by sliding the curved bar 206 in one of the two opposing directions of the curved channel axis C-C until no part of the curved bar 206 remains located within the curved channel 106.

When the first and second components 100, 200 are coupled together as described above, the terminal surface 211 of the curved bar 206 is in surface contact with the floor surface 107 of the curved channel 106. Furthermore, the curved end surface 207 of the second component 200 is in surface contact with the terminal surfaces 110 of the curved walls 108 of the first component 100. In the exemplified embodiment, the terminal surface 211, the floor surface 107, the curved end surface 207, and the terminal surfaces 110 are all smooth surfaces that are preferably free of undulations to enable the first and second components 100, 200 to freely slide relative to one another when the locking element/set screws 210 are not being used to lock the first and second components 100, 200 into a particular relative position. While the first and second components 100, 200 are able to freely slide in the direction of the curved channel axis C-C, the first and second components 100, 200 cannot be separated in a direction perpendicular to the curved channel axis C-C due to the dove-tail shapes of the curved bar 206 and the curved channel 106.

Once the curved bar 206 is located within the curved channel 106, the curved bar 206 can be slid along the curved channel axis C-C until the curved bar 206 is located at a desired position within the curved channel 106. Thus, the curved bar 206 is slidable within the curved channel 106 so as to be slid along the curved channel axis C-C in first and second opposite directions so that the first and second components 100, 200 can be selectively adjusted to a plurality of relative positions.

Upon the desired location/relative position being reached, the locking element is used to lock the first and second components 100 in the desired relative positions.

Specifically, in the exemplified embodiment the first and second components 100, 200 are locked together by inserting the one or more set screws 210 within the through holes 197 formed into the curved walls 108 (see FIG. 7B). The one or more set screws 210 are then tightened until the terminal end of the set screws 210 are in contact with the curved bar 206 that is positioned within the curved channel 106, thereby locking the first and second members 100, 200 together to prohibit further translation of the curved bar 206 within the curved channel 106 in the direction of the curved channel axis C-C. Specifically, the locking element/set screws 210 prevents the first and second components 100, 200 from being moved relative to one another in the first and second opposing directions of the curved channel axis C-C. As a result, the first and second members 100, 200 can be locked in a desired relative position to achieve a desired offset (if any). The set screw 210 can be loosened so that the curved bar 206 can again slide within the curved channel 106 so that adjustments can be made. Stated simply, the set screw 210 allows for selective positioning of the second component 200 relative to the first component 100. Thus, the locking element (or set screws 210) are alterable between (1) a first state in which the locking element does not prohibit the curved bar 206 from sliding within the curved channel 106; and (2) a second state in which the locking element locks the first and second components in a specific relative position.

Referring now to FIGS. 8A-C and 9 concurrently, the movement of the first and second components 100, 200 relative to one another will be further described. When the curved bar 206 is located within the curved channel 106, the curved bar 206 remains slidable within the curved channel 106 along the curved channel axis C-C in first and second opposite directions (i.e., in a direction towards each of the opposing lateral sides 105 of the first component 100) so long as the set screws 210 or other locking mechanism are not preventing such movement. Sliding the curved bar 206 within the curved channel 106 results in the first and second components 100, 200 being selectively adjusted to a plurality of relative positions.

FIG. 8A illustrates the first and second components 100, 200 in a first relative position in which the first and second central axes A-A, B-B are parallel to one another. In this relative positioning, the second component 200 is in an aligned position with respect to the first component 100 so that the first and second central axes A-A, B-B of the first and second members 100, 200 are substantially coaxial with one another. The locking element or set screw 210 can be tightened to lock the first and second components 100, 200 into this position, or loosened to enable the first and second components 100, 200 to be moved relative to one another. FIG. 8B illustrates the first and second components 100, 200 in a second relative position in which the first and second axes A-A, B-B are angularly offset from one another by an angle 01. FIG. 8C illustrates the first and second components 100, 200 in a third relative position in which the first and second axes A-A, B-B are angularly offset from one another by an angle 02. Simply by sliding the curved bar 206 within the curved channel 106, the angle between the first and second axes A-A, B-B can be modified. The locking element can be set into the locked state or the set screw 210 can be tightened to prevent relative movement between the second component 200 and the first component 100 in any position as desired (including the position whereby the second axis B-B is offset from the first axis A-A by the angle 01 or the angle 02 or any other angle).

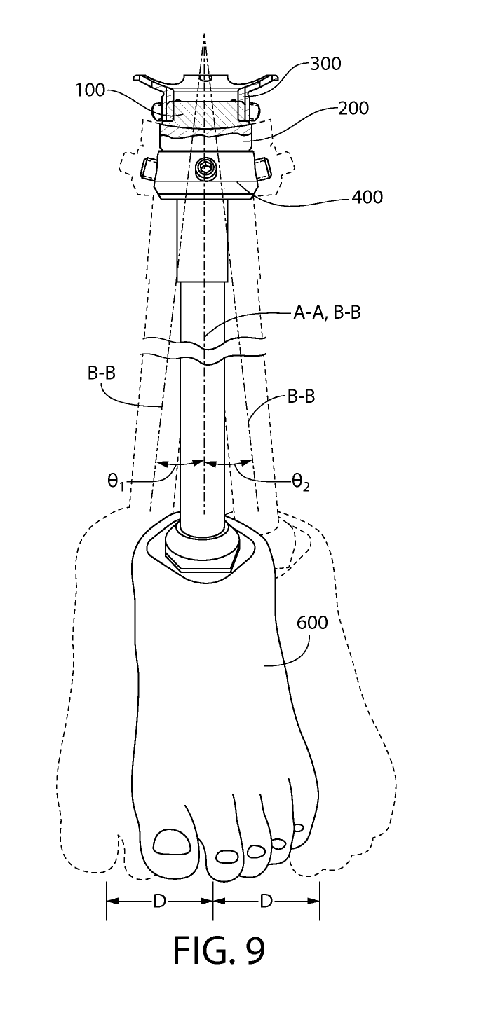

Referring to FIG. 9, moving the curved bar 206 within the curved channel 106 changes an offset distance D at the end of the prosthetic 600. Specifically, due to the curvature of the curved channel 106 and the curved bar 206, a minor adjustment of the position of the second component 200 relative to the first component 100 (via sliding of the curved bar 206 within the curved channel 106) can result in an increased offset distance D at the end of the prosthetic 600 that could not be achieved by previous devices. This results because the curved nature of the curved bar 206 and the curved channel 106 causes an angular offset of the first central axis A-A of the first component 100 and the second central axis B-B of the second component 200 when the curved bar 206 is slid/translated only a small distance relative to the curved channel 106. This is achieved more specifically due to the component that is coupled to the prosthesis having a concave surface and the component that is coupled to the residual limb having a convex surface, and can also be achieved if the component coupled to the prosthesis has a convex surface and the component that is coupled to the residual limb has a concave surface.

As a result of the curved nature of the abutting surfaces of the first and second components 100, 200 noted herein, a small translational movement of the first and second components 100, 200 relative to one another in the direction of the curved channel axis C-C achieves a greater degree of movement of the prosthetic 600 than if the curved abutting surfaces were flat. Thus, there is a small portion of the first and second components 100, 200 that are jutting out relative to one another despite potentially having a large offset distance D (see FIGS. 8B and 8C).

It should be appreciated that the prosthetic coupling apparatus 1000 can be used to adjust the positioning of an artificial limb/prosthetic in the lateral or medial directions or in the anterior or posterior directions, or anything in between depending on the positioning of the curved groove 106 and curved bar 206 relative to the prosthetic.

Referring now to FIGS. 10-12B, a prosthetic coupling apparatus 1000A according to a second embodiment of the present invention is illustrated. The prosthetic coupling apparatus 1000A is identical to the prosthetic coupling apparatus 1000 discussed above with the exception that the pyramid block 203 of the second component 200 has been replaced with a pyramid block receiving cavity 230A. All features of the prosthetic coupling apparatus 1000 are applicable to the prosthetic coupling apparatus 1000A except that the pyramid block 203 has been changed to the pyramid receiving cavity 203A. Thus, the description of the features and components of the prosthetic coupling apparatus 1000 that are also present in the prosthetic coupling apparatus 1000A (whether marked with a reference numeral or not) is applicable to the prosthetic coupling apparatus 1000A. The details with regard to the contours and curvatures of the various surfaces of the prosthetic coupling apparatus 1000A are not described herein, it being understood that the description of those features from the prosthetic coupling apparatus 1000 applies.

Specifically, the prosthetic coupling apparatus 1000A comprises a first component 100A and a second component 200A. The first component 100A comprises a first end portion 101A and a second end portion 102A and the second component 200A comprises a first end portion 201A and a second end portion 202A. The first end portion 101A of the first component comprises a hub 103A configured to be inserted within and coupled to a collar 302A of a third component, such as a cup member 300A. The second end portion 102A of the first component 100A comprises a curved channel 106A that is identical to the curved channel 106 of the first component 100 of the prosthetic coupling apparatus 1000.

The second end portion 202A of the second component 200A comprises a curved bar 206A that is identical to the curved bar 206 of the second component 200 of the prosthetic coupling apparatus 1000 described above. As noted above, the only difference between the prosthetic coupling apparatus 1000 and the prosthetic coupling apparatus 1000A is that with the prosthetic coupling apparatus 1000A, the first end portion 201A of the second component 200A comprises a pyramid block receiving cavity 203A instead of a pyramid block. Forming the first end portion 201A of the second component 200A with the pyramid block receiving cavity 203A enables the second component 200A to be coupled to a prosthetic component that has a pyramid block as a connecting feature. Thus, as noted above, the prosthetic coupling apparatus 1000, 1000A can include various different connecting features while still having the novel features of the first and second components with corresponding/slidingly mating curved channels 106, 106A and curved bars 206, 206A.

The pyramid block receiving cavity 203A is a cavity or hollow space that is formed into the second end portion 202A of the second component 200A. The pyramid block receiving cavity 203A is defined by a sidewall 219A that has a first portion that converges from the terminal end of the second end portion 202A of the second component 200A to a transition point, and a second portion that diverges from the transition point to the floor of the pyramid block receiving cavity 203A. Furthermore, a plurality of through holes 220A are formed into the second component 200A and set screws 221A are positioned within the through holes 220A. Thus, the second end portion 202A of the second component 200A can be coupled to a prosthetic component by inserting a pyramid block of the prosthetic component into the pyramid block receiving cavity 203A of the second component 200A, and then tightening the set screws 221A to prevent the pyramid block from being removed from the pyramid block receiving cavity 203A.

Referring now to FIGS. 13, a prosthetic coupling apparatus 1000B according to a third embodiment of the present invention is illustrated. The prosthetic coupling apparatus 1000B is identical to the prosthetic coupling apparatus 1000 discussed above with the exception that the pyramid block 203 has been replaced with a pyramid receiver 203A similar to that discussed above in FIGS. 10-12 with regard to the prosthetic coupling apparatus 1000A and the hub 103 has been replaced with a pyramid block receiving cavity 103B. Thus, all features of the prosthetic coupling apparatus 1000 are applicable to the prosthetic coupling apparatus 1000B except that the pyramid block 203 has been changed to the pyramid block receiving cavity 203B and the hub 103 has been replaced with the pyramid block receiving cavity 103B. Thus, the description of the features and components of the prosthetic coupling apparatus 1000 that are also present in the prosthetic coupling apparatus 1000B (whether marked with a reference numeral or not) is applicable to the prosthetic coupling apparatus 1000B. The details with regard to the contours and curvatures of the various surfaces of the prosthetic coupling apparatus 1000B are not described herein, it being understood that the description of those features from the prosthetic coupling apparatus 1000 applies.

The prosthetic coupling apparatus 1000B comprises a first component 100B and a second component 200B. The first component 100B comprises a first end portion 101B and a second end portion 102B and the second component 200B comprises a first end portion 201B and a second end portion 202B. The first end portion 101B of the first component 100B comprises the pyramid block receiving cavity 103B configured to be coupled to a prosthetic component. The second end portion 102B of the first component 100B comprises a curved channel 106B that is identical to the curved channel 106 of the first component 100 of the prosthetic coupling apparatus 1000.

The second end portion 202B of the second component 200B comprises a curved bar 206B that is identical to the curved bar 206 of the second component 200 of the prosthetic coupling apparatus 1000 described above. Furthermore, the first end portion 201B of the second component 200B comprises a pyramid block receiving cavity 203B. Thus, in this embodiment each of the first end portions 101B, 201B of the first and second components 100B, 200B comprises a pyramid block receiving cavity 103B, 203B. The structures and shapes of the pyramid block receiving cavities 103B, 203B are identical to one another and to the pyramid block receiving cavity 203A described above with reference to FIGS. 10-12.

Forming the first end portions 101B, 201B of the first and second components 100B, 200B with the pyramid block receiving cavities 103B, 203B enables each of the first and second components 100B, 200B to be coupled to a prosthetic component that has a pyramid block as a connecting feature. Thus, as noted above, the prosthetic coupling apparatus 1000, 1000A, 100B can be various different connecting features while still having the novel features of the first and second components with corresponding/slidingly mating curved channels 106, 106A, 106B and curved bars 206, 206A, 206B. Furthermore, regardless of the connecting feature on the first and second components 100, 100A, 100B, 200, 200A, 200B, each of the first and second components 100, 100A, 100B, 200, 200A, 200B is in certain embodiments a monolithic integrally formed singular component.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly.

What is claimed is:

1. A prosthetic alignment device comprising:
a first component extending along a first central axis comprising:
a first end portion configured to be coupled to one of a prosthesis or a residual limb; and
a second end portion comprising a curved channel extending along a curved channel axis between opposite lateral side surfaces of the first component;
a second component extending along a second central axis comprising:
a first end portion configured to be coupled to the other one of the prosthesis or the residual limb; and
a second end portion comprising a curved bar that extends along a curved bar axis;
the curved bar slidably disposed within the curved channel to couple the first and second components together, the curved bar slidable within the curved channel so as to be slid along the curved channel axis in first and second opposite directions so that the first and second components can be selectively adjusted to a plurality of relative positions, the plurality of relative positions comprising: (1) a first relative position in which the first and second central axes are parallel to one another; and (2) a second relative position in which the first and second central axes are angularly offset from one another.

2. The prosthetic alignment device of claim 1 further comprising:
one of the first end portion of the first component or the first end portion of the second component comprising a hub configured to be inserted within and coupled to a collar of a third component;
the other one of the first end portion of the first component or the first end portion of the second component comprising a pyramid block; and
wherein each of the first and second components is a monolithic integrally formed singular component.

3. The prosthetic alignment device of claim 1 further comprising:
one of the first end portion of the first component or the first end portion of the second component comprising a hub configured to be inserted within and coupled to a collar of a third component;
the other one of the first end portion of the first component or the first end portion of the second component comprising a pyramid block receiving cavity; and
wherein each of the first and second components is a monolithic integrally formed singular component.

4. The prosthetic alignment device of claim 1 further comprising:
each of the first end portions of the first and second components comprising a pyramid block receiving cavity; and
wherein each of the first and second components is a monolithic integrally formed singular component.

5. The prosthetic alignment device of claim 1 wherein each of the channel and bar axes have a substantially constant radius of curvature along their entire length.

6. The prosthetic alignment device of claim 1 further comprising:
the curved channel axis intersecting the first central axis at a first intersection point, wherein a line tangent to the curved channel axis at the first intersection point is substantially perpendicular to the first central axis; and
the curved bar axis intersecting the second central axis at a second intersection point, wherein a line tangent to the curved bar axis at the second intersection point is substantially perpendicular to the second central axis.

7. The prosthetic alignment device of claim 1 wherein the first and second central axes are coaxial with one another when the first and second components are in the first relative position.

8. The prosthetic alignment device of claim 1 wherein the bar has a curved terminal surface and the curved channel has a curved floor surface, wherein the curved terminal surface, the curved floor surface, the curved bar axis, and the curved channel axis have substantially equal radii of curvature.

9. The prosthetic alignment device of claim 1 further comprising a locking element operably coupled to the first component, the locking element alterable between: (1) a first state in which the locking element does not prohibit the curved bar from sliding within the curved channel; and (2) a second state in which the locking element locks the first and second components in a selected one of the plurality of relative positions.

10. The prosthetic alignment device of claim 1 wherein the first end portion of the first component comprises first and second wall portions that define walls of the curved channel, each of the first and second wall portions having a curved terminal surface; wherein the curved bar protrudes from a curved end surface of the second end portion of the second component; and wherein the curved terminal surfaces of the wall portions and the curved end surfaces of the second end portion of the second component have substantially equal radii of curvature.

11. The prosthetic alignment device of claim 1 wherein the curved bar has a transverse cross-sectional profile that mates with a transverse cross-sectional profile of the curved channel so that the first and second components cannot be separated from one another without sliding the curved bar along the curved channel axis until the curved bar is located entirely outside of the curved channel.

12. The prosthetic alignment device of claim 11 wherein each of the curved bar and the curved channel have a dove-tail transverse cross-sectional profile.

13. A prosthetic alignment device comprising:
a first component comprising:
a first end portion configured to be coupled to one of a prosthesis or a residual limb; and
a second end portion comprising a curved channel extending along a curved channel axis;
a second component extending along a second central axis comprising:
a first end portion configured to be coupled to the other one of the prosthesis or the residual limb; and
a second end portion comprising a curved bar that extends along a curved bar axis;
the curved bar positioned within the curved channel so as to be slidable along the curved channel axis in first and second opposite directions so that the first and second components can be selectively adjusted to a plurality of relative positions.

14. The prosthetic alignment device of claim 13 further comprising:
one of the first end portion of the first component or the first end portion of the second component comprising a hub configured to be inserted within and coupled to a collar of a third component;
the other one of the first end portion of the first component or the first end portion of the second component comprising a pyramid block; and
wherein each of the first and second components is a monolithic integrally formed singular component.

15. The prosthetic alignment device of claim 13 further comprising:
one of the first end portion of the first component or the first end portion of the second component comprising a hub configured to be inserted within and coupled to a collar of a third component;
the other one of the first end portion of the first component or the first end portion of the second component comprising a pyramid block receiving cavity; and
wherein each of the first and second components is a monolithic integrally formed singular component.

16. The prosthetic alignment device of claim 13 further comprising:
each of the first end portions of the first and second components comprising a pyramid block receiving cavity; and
wherein each of the first and second components is a monolithic integrally formed singular component.

17. The prosthetic alignment device of claim 13 further comprising a locking element operably coupled to the first component, the locking element alterable between: (1) a first state in which the locking element does not prohibit the curved bar from sliding within the curved channel; and (2) a second state in which the locking element locks the first and second components in a selected one of the plurality of relative positions.

18. The prosthetic alignment device of claim 1 wherein the curved bar has a transverse cross-sectional profile that mates with a transverse cross-sectional profile of the curved channel so that the first and second components cannot be separated from one another without sliding the curved bar along the curved channel axis until the curved bar is located entirely outside of the curved channel.

19. The prosthetic alignment device of claim 18 wherein each of the curved bar and the curved channel have a dove-tail transverse cross-sectional profile.

20. A prosthetic alignment device comprising:
a first monolithic component extending along a first central axis, the first component consisting of:
a first end portion configured to be coupled to one of a prosthesis or a residual limb; and
a second end portion comprising a curved channel extending along a curved channel axis between opposite lateral side surfaces of the first component;
a second monolithic component extending along a second central axis consisting of:
a first end portion configured to be coupled to the other one of the prosthesis or the residual limb; and
a second end portion comprising a curved bar that extends along a curved bar axis;
the curved bar slidably disposed within the curved channel to couple the first and second components together, the curved bar slidable within the curved channel so as to be slid along the curved channel axis in first and second opposite directions so that the first and second components can be selectively adjusted to a plurality of relative positions, the plurality of relative positions comprising: (1) a first relative position in which the first and second central axes are parallel to one another; and (2) a second relative position in which the first and second central axes are angularly offset from one another
wherein each of the first and second components is a monolithic integrally formed singular component;
wherein each of the channel and bar axes have a substantially constant radius of curvature along their entire length;
the curved channel axis intersecting the first central axis at a first intersection point, wherein a line tangent to the curved channel axis at the first intersection point is substantially perpendicular to the first central axis;
the curved bar axis intersecting the second central axis at a second intersection point, wherein a line tangent to the curved bar axis at the second intersection point is substantially perpendicular to the second central axis;
the curved bar has a curved terminal surface and the curved channel has a curved floor surface, wherein the curved terminal surface, the curved floor surface, the curved bar axis, and the curved channel axis have substantially equal radii of curvature; and
a locking element operably coupled to the first component, the locking element alterable between: (1) a first state in which the locking element does not prohibit the curved bar from sliding within the curved channel; and (2) a second state in which the locking element locks the first and second components in a selected one of the plurality of relative positions.
